(19) United States
(12) Patent Application Publication
(10) Pub. No.: US 2019/0120782 A1
(43) Pub. Date: Apr. 25, 2019

(54) NANOBIOSENSOR FOR DETECTING ALLERGIES, MANUFACTURING METHOD THEREFOR, AND DETECTION SYSTEM COMPRISING SAME

(71) Applicant: SANGMYUNG UNIVERSITY CHEONAN COUNCIL FOR INDUSTRY ACADEMIC COOPERATION, Cheonan-si, Chungcheongnam-do (KR)

(72) Inventors: Jin Young LEE, Seoul (KR); Jun Hyun OH, Daegu (KR); Hyun Kyung CHOI, Incheon (KR)

(73) Assignee: SANGMYUNG UNIVERSITY CHEONAN COUNCIL FOR INDUSTRY ACADEMIC COOPERATION, Cheonan-si, Chungcheongnam-do (KR)

(21) Appl. No.: 16/302,952

(22) PCT Filed: Dec. 8, 2016
(86) PCT No.: PCT/KR2016/014371

§ 371 Date: Nov. 19, 2018

(30) Foreign Application Priority Data
May 19, 2016 (KR) 10-2016-0061325

(51) Int. Cl. G01N 27/12; G01N 33/02; G01N 33/68; G01N 33/544

(52) U.S. Cl. CPC G01N 27/127; G01N 33/544; G01N 33/68; G01N 33/02

(57) ABSTRACT

The present invention provides a biosensor for detecting allergies. The biosensor comprises a substrate; an electrode layer formed on a portion of an upper side of the substrate; a single-walled carbon nanotube layer formed on another portion of the upper side of the substrate; a linker layer formed on the single-walled carbon nanotube layer; and an antibody which is immobilized and bonded to the linker layer. In the biosensor for detecting allergies according to the present invention, 1-pyrenebutanoic acid succinimidyl ester is comprised as a linker on the substrate which is coated with a single-walled carbon nanotube, and the antibody which is capable of capturing an allergy-inducing protein is immobilized on the linker. When the allergy-inducing protein is captured by the antibody, a resistance value is changed, so that the allergy-inducing protein is capable of being detected with high sensitivity in a short time by sensing the changed resistance value.

### TECHNICAL FIELD

The present invention relates to a nanobiosensor for detecting allergies, a method of fabricating the same, and an allergy detection system comprising the same.

### BACKGROUND ART

Recently, due to the transition to an aging society and a modern society, the proportion of diseases, such as circulatory organ system diseases, nervous system diseases, allergies, and obesity, caused by ingestion of high-nutrient foods or environmental pollution is increasing. Among them, the incidence of allergy-related diseases is increasing daily due to changes in eating habits and severity of pollution.

Allergy-related diseases exhibit various symptoms such as asthma, allergic dermatitis, atopic dermatitis, allergic conjunctivitis, and allergic enteritis, and the most common examples thereof may include asthma and allergic rhinitis, which are respiratory diseases and atopic dermatitis, which is a skin disease.

Bronchodilators, antihistamines, antispasmodic drugs, and steroids have been used as medicines for allergy-related diseases. In recent years, however, disodium cromoglycate, tranilast, ketotifen, and azelastine, etc. having inhibitory activity and antagonistic activity with respect to releasing chemical mediator have been commercially available. Research and development for anti-allergic materials has been carried out, but it is still insufficient.

Currently, it is known that allergic reactions are caused by food allergens. Accordingly, there is increasing interest in a method of preventing induction of allergies by detecting food allergens. The food allergens are detected not only in food itself but also in processed food which is prepared using the food as a raw material.

Conventionally, in order to examine allergy-inducing foods, a method of directly detecting the protein inducing allergies using an enzyme immunoassay (EIA) or an enzyme-linked immunosorbent assay (ELISA) is used. However, since the proteins to be examined are most likely to be destroyed in the preparing process or in many processing steps with the mixing of various raw ingredients, there is a problem of low sensitivity and low specificity of the method.

Further, a method of detecting DNA of foods inducing allergies using a polymerase chain reaction (PCR) has been used as another detection method. The above method is advantageous in that DNA can be detected even in foods with various steps, such as heating and processing, etc., compared to the conventional protein detection method. However, there is a drawback in that time-consuming and equipment-requiring method for protein detection leads to low efficiency.

Therefore, it is required to establish a method of easily and simply detecting allergy-inducing proteins in food.

### DISCLOSURE

### Technical Problem

Accordingly, the present invention has been made to solve the problems occurring in the prior art, and an object of the present invention is to provide a technical content of a carbon-nanotube-based nanobiosensor for detecting allergies, which is capable of detecting an allergy-inducing protein with high sensitivity and which enables field detection practically when applied to the food industry.

### Technical Solution

In order to accomplish the above object, the present invention provides a nanobiosensor for detecting allergies. The nanobiosensor comprises a substrate; an electrode layer formed on a portion of an upper side of the substrate; a single-walled carbon nanotube layer formed on another portion of the upper side of the substrate; a linker layer formed on the single-walled carbon nanotube layer; and an antibody which is bonded to the linker layer using an immobilization reaction and which is capable of capturing an allergy-inducing material.

Further, the substrate is a chromium (Cr)-doped silicon substrate.

Further, the electrode layer comprises gold (Au).

Further, the linker layer comprises 1-pyrenebutanoic acid succinimidyl ester.

Further, the antibody captures one or more peanut-allergy-inducing proteins selected from the group consisting of a vicilins-based protein, a conglutins-based protein, and a glycinins-based protein.

The present invention also provides a method of fabricating a nanobiosensor for detecting allergies. The method comprises (a) forming an electrode pattern on a substrate; (b) forming a single-walled carbon nanotube layer by applying a single-walled carbon nanotube on the substrate on which the electrode pattern is formed; (c) forming a linker layer by applying 1-pyrenebutanoic acid succinimidyl ester on the single-walled carbon nanotube layer; and (d) bonding an antibody to the linker layer by applying a mixed solution containing the antibody for capturing an allergy-inducing material on the single-walled carbon nanotube layer on which the linker layer is formed.

Further, in (a) the forming the electrode pattern, a gold (Au) electrode pattern is formed using an e-beam evaporation method (e-beam evaporator).

Further, in (b) the forming the single-walled carbon nanotube layer, the mixed solution containing the single-walled carbon nanotube at a concentration of 0.1 to 10 mg/mL is applied.

Further, in (d) the bonding the antibody to the linker layer, the mixed solution containing the antibody capable of capturing peanut-allergy-inducing proteins is applied on the single-walled carbon nanotube layer on which the linker layer is formed, thus forming an antibody layer.

Further, in (d) the bonding the antibody to the linker layer, the mixed solution containing the antibody at a concentration of 0.001 to 0.003 mg/mL is applied on the single-walled carbon nanotube layer on which the linker layer is formed.

The present invention also provides an allergy detection system which comprises the nanobiosensor for detecting allergies as described above, a resistance-sensing unit electrically connected to the nanobiosensor to thus sense a change in the resistance value of the nanobiosensor, and a display unit displaying the change in resistance value sensed by the resistance-sensing unit.

### Advantageous Effects

In a nanobiosensor for detecting allergies according to the present invention, 1-pyrenebutanoic acid succinimidyl ester is comprised as a linker on a substrate which is coated with a single-walled carbon nanotube, and an antibody which is capable of capturing an allergy-inducing material is immobilized on the linker. When a sample comprising the allergy-inducing material is supplied, the antibody captures the allergy-inducing material to thus change a resistance value. It is possible to detect the allergy-inducing material with high sensitivity in a short time by sensing the changed resistance value.

In particular, the above-described nanobiosensor for detecting allergies comprises an antibody which captures a peanut-allergy-inducing protein such as an anti-vicilin-based protein, an anti-conglutin-based protein, or an anti-glycinin-based protein. Accordingly, the peanut-allergy-inducing protein can be being sensed with high sensitivity even when supplied with a sample containing a small amount of the peanut-allergy-inducing protein in ng/mL level. Therefore, it is possible to effectively use the nanobiosensor of the present invention as a portable nanobiosensor for detecting a peanut allergy.

### DESCRIPTION OF DRAWINGS

FIG. 1 is a conceptual view mimetically showing a nanobiosensor for detecting allergies according to the present invention;

FIG. 2 is a conceptual view mimetically showing an allergy detection system according to the present invention;

FIG. 3 is a graph showing the results of an antigen-antibody reaction depending on a change in the concentration of a peanut-allergy-inducing protein using the nanobiosensor fabricated by a method according to an embodiment;

FIG. 4 is a graph showing changes in the electrical resistance of a substrate (SWCNT) in which a carbon nanotube is immobilized, a substrate (SWCNT+linker) in which a carbon nanotube and a linker are immobilized, and a nanobiosensor (SWCNT+linker+Ab) in which a carbon nanotube, a linker, and an antibody are immobilized, fabricated by the method according to the embodiment;

FIG. 5 is a graph showing changes in electrical resistance of a nanobiosensor (SWCNT+linker+Ab) fabricated by the method according to the embodiment and a control group, depending on the presence or absence of a peanut-allergy-inducing protein; and

FIG. 6 shows the detection curve for each concentration of the peanut-allergy-inducing protein supplied to the nanobiosensor fabricated by the method according to the embodiment.

### BEST MODE

Hereinafter, the present invention will be described in detail.

The present invention provides a nanobiosensor for detecting allergies, comprising a substrate, an electrode layer formed on a portion of the upper side of the substrate, a single-walled carbon nanotube layer formed on another portion of the upper side of the substrate, a linker layer formed on the single-walled carbon nanotube layer, and an antibody which is bonded to the linker layer using an immobilization reaction and which is capable of capturing an allergy-inducing material.

The substrate may comprise any material without limitation as long as the material is capable of being commonly used for forming an electrode, and representative examples thereof may be a silicon substrate.

As the silicon substrate, a chromium (Cr)-doped silicon substrate may be preferably used for the purpose of joining a single-walled carbon nanotube formed in a step to be described later. A gold electrode may be formed on the chromium-doped silicon substrate to thus form a substrate having a structure in which chromium and gold are deposited. Accordingly, the single-wall carbon nanotube and silicon may be joined to each other, so that a change in resistance value by an impedance change is sensed, thereby forming a nanobiosensor having high selectivity to an allergy-inducing protein. Therefore, the electrode layer may be a gold electrode layer comprising gold (Au).

In addition, since the single-walled carbon nanotube has high sensitivity, when the antibody and the allergy-inducing protein are bonded to each other, the resistance value is sensitively changed, so that the single-walled carbon nanotube may be effectively used in the nanobiosensor.

Further, the linker layer comprises 1-pyrenebutanoic acid succinimidyl ester. Due to Π-Π stacking of the hydrophobic pyrenyl group of 1-pyrenebutanoic acid on the hydrophobic wall of the single-walled carbon nanotube layer and an electrostatic interaction thereof, 1-pyrenebutanoic acid is immobilized onto the single-walled carbon nanotube layer, and the linker layer that provides a site to which the antibody is bonded may be formed on the substrate.

As the above-described antibody, any antibody may be used without limitation as long as the antibody captures various genes, proteins, enzymes, peptides, amino acids, or aptamers which are allergy-inducing materials inducing allergies. For example, when a sample containing an allergy-inducing material is added, the antibody and the allergy-inducing material may be bonded through a noncovalent bonding reaction, so that the allergy-inducing material is captured by the antibody. Preferably, the antibody capable of capturing an allergy-inducing protein may be used.

For example, as the antibody, it is preferable to use an anti-vicilin antibody, an anti-conglutin antibody, an anti-glycinin antibody, or a mixture antibody thereof which is capable of capturing vicilin-based proteins, conglutin-based proteins, and glycinin-based proteins, which are proteins that induce a peanut allergy when they enter a human body through a method such as ingestion or injection. More preferably, an anti-Ara h1 antibody capable of capturing an Ara h1 protein, which is a kind of the vicilin-based protein, may be used.

The nanobiosensor for detecting allergies according to the present invention comprises 1-pyrenebutanoic acid succinimidyl ester as a linker on the substrate coated with the single-walled carbon nanotube. The linker may serve to immobilize an antibody capable of capturing an allergy-inducing protein, and a change in resistance value caused when the antibody captures the allergy-inducing protein may be sensed, thereby sensing the allergy-inducing protein with high sensitivity in a short time.

In particular, the above-described nanobiosensor for detecting allergies may comprise an anti-vicilin antibody, an anti-conglutin antibody, an anti-glycinin antibody, or a mixture antibody thereof, which captures peanut-allergy-inducing proteins, as the antibody, so that the peanut-allergy-inducing proteins are captured with high sensitivity even when supplied with an allergy-inducing protein sample in ng/mL level. Accordingly, the nanobiosensor is capable of being more effectively used as a portable nanobiosensor for detecting a peanut allergy.

Further, the present invention provides a method of fabricating a nanobiosensor for detecting allergies. The method comprises (a) forming an electrode pattern on a substrate; (b) forming a single-walled carbon nanotube layer by applying a single-walled carbon nanotube on the substrate on which the electrode pattern is formed; (c) forming a linker layer by applying 1-pyrenebutanoic acid succinimidyl ester on the single-walled carbon nanotube layer; and (d) bonding an antibody to the linker layer by applying a mixed solution containing the antibody for capturing an allergy-inducing material on the single-walled carbon nanotube layer on which the linker layer is formed.

The step (a) is a step of forming the electrode pattern on the substrate. For the purpose of joining the formed single-walled carbon nanotube, a chromium (Cr)-doped silicon substrate may be preferably used, so that a gold electrode is formed on the chromium-doped silicon substrate, thus forming a substrate having a structure in which chromium and gold are deposited. Accordingly, the single-walled carbon nanotube and silicon may be joined each other so that a change in resistance value due to an impedance change is sensed, thus fabricating a nanobiosensor for detecting allergies with high selectivity.

In order to form the metal pattern, a mask for forming the electrode pattern is prepared, the mask is placed on the upper side of the substrate, and gold (Au) is deposited on the substrate using an e-beam lithography process, so that chromium and gold may be deposited on the substrate, thereby forming an electrode pattern having high selectivity.

Further, the distance between gold electrodes may be set to about 0.01 to 0.2 cm, and a gold electrode pattern may be formed using the e-beam lithography under a vacuum pressure condition.

The step (b) is a step of forming the single-walled carbon nanotube layer by applying the single-walled carbon nanotube on the substrate on which the electrode pattern is formed. In the present step, a single-walled carbon nanotube with high sensitivity is used. In order to apply the single-walled carbon nanotube, a mixed solution containing the single-walled carbon nanotubes that are uniformly dispersed may be applied on the substrate using a solution process, thus forming the single-walled carbon nanotube layer on the substrate.

In order to form the single-walled carbon nanotube, it is preferable to use the mixed solution containing the single-walled carbon nanotube at a concentration of 0.1 to 10 mg/mL. When the concentration of the mixed solution is less than 0.1 mg/mL, the sensitivity of the nanobiosensor may be reduced due to the high resistance value of the nanobiosensor. When the concentration is more than 10 mg/mL, since the resistance value is not reduced, the single-walled carbon nanotube layer may be formed using the mixed solution having the above-described concentration. In the present step, after the single-walled carbon nanotube layer is formed, although the surface of the substrate is repeatedly washed using deionized water three times or more to thus remove impurities, a stable single-walled nanotube layer having a resistance value which is not significantly changed may be formed. More preferably, the single-walled carbon nanotube may be formed on the substrate using a mixed solution with a concentration of 0.1 mg/mL.

Moreover, in the present step, after the mixed solution containing the single-walled carbon nanotube is applied on the upper side of the substrate, current may be applied in the state in which the mixed solution is applied on the upper side of the substrate so that a single-walled carbon nanotube layer aligned in a predetermined direction is formed. The alignment of the single-walled carbon nanotube layer which is aligned as described above is improved, so that the increase in the resistance value may be enhanced when an allergy sample is bonded to the antibody, thus sensing a target allergy-inducing material with high sensitivity.

The step (c) is a step of forming the linker layer by applying 1-pyrenebutanoic acid succinimidyl ester on the single-walled carbon nanotube layer.

To be more specific, due to Π-Π stacking of the hydrophobic pyrenyl group of 1-pyrenebutanoic acid succinimidyl ester on the hydrophobic wall of the single-walled carbon nanotube layer and electrostatic interaction thereof, 1-pyrenebutanoic acid may be immobilized on the single-walled carbon nanotube layer, thus forming the linker layer on the substrate.

The step (d) is a step of bonding the antibody to the linker layer by applying the mixed solution containing the antibody for capturing the allergy-inducing material on the single-walled carbon nanotube layer on which the linker layer is formed. By applying an antibody capable of capturing the gene, protein, peptide, amino acid, and aptamer of a target allergy-inducing material on the single-walled carbon nanotube layer on which the linker layer is formed as described above, the nanobiosensor capable of capturing the target allergy-inducing material that is present in the sample may be fabricated using the antibody capable of capturing an allergy when the allergy sample is supplied.

To be more specific, the antibody bonded to the linker layer has a double structure comprising a variable region and a constant region. The constant region of the antibody is immobilized to the linker layer through a nucleophilic substitution reaction with an amine group, thus being immobilized on the surface of the linker layer. Thus, the nanobiosensor may be fabricated so that the variable region capable of capturing the target allergy-inducing material comprised in the sample containing the allergy-inducing material is exposed to the outside.

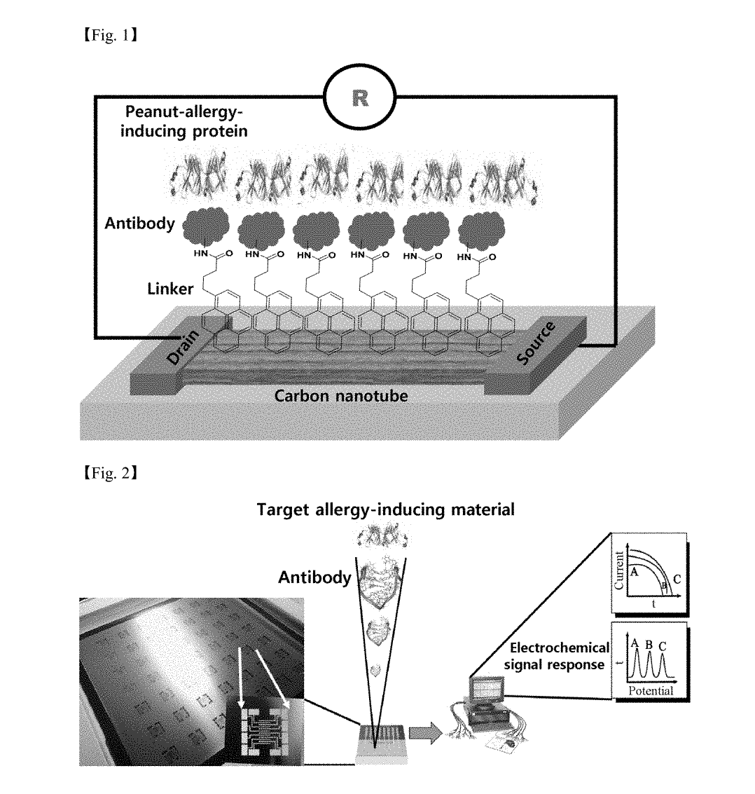

In the nanobiosensor having the structure containing the single-walled carbon nanotube, the linker layer, and the antibody as described above, the linker layer and the antibody may be bonded to each other, whereby a nanobiosensor in which the resistance value of the single-walled carbon nanotube is not significantly changed but is constant may be fabricated (see FIG. 1).

The nanobiosensor prepared as described above may comprise a coloring material that develops a color when the target allergy-inducing material is captured, so that whether the target allergy-inducing material is present are confirmed and the concentration of the target allergy-inducing material is quantified using the presence or absence of color development by a color-developing unit. Moreover, in order to fabricate a highly sensitive nanobiosensor when a food sample containing a target allergy at a low concentration is supplied, the present invention is composed so that the target allergen is sensed by a change in resistance value caused when the target allergy-inducing material is bonded to the antibody of the nanobiosensor. When the sample containing the allergy-inducing material is supplied to the nanobiosensor, the target allergy-inducing material comprised in the sample may be captured by the variable region of the antibody of the nanobiosensor, which rapidly changes the resistance value of the single-walled carbon nanotube, so that the target allergy-inducing material included in the sample may be sensed with high sensitivity by a resistance sensor provided in order to sense the change in resistance value.

In the present step, the mixed solution containing the antibody at a concentration of 0.001 to 0.003 mg/mL may be applied on the single-walled carbon nanotube layer on which the linker layer is formed, whereby the resistance value of the nanobiosensor is prevented from being rapidly increased, and the nanobiosensor sensing the target allergy-inducing material with high sensitivity is fabricated. More preferably, a mixed solution containing the antibody at a concentration of 0.003 mg/mL may be applied.

For example, in the present step, an allergy-inducing protein comprised in food may be set as the target allergy-inducing material, and the mixed solution containing the antibody capable of capturing the allergy-inducing protein may be applied on the single-walled carbon nanotube layer on which the linker layer is formed, thus fabricating a nanobiosensor capable of detecting the allergy-inducing protein comprised in food with high sensitivity.

As described above, in the method of fabricating the nanobiosensor for detecting allergies according to the present invention, a noncovalent immobilization process in which 1-pyrenebutanoic acid succinimidyl ester is bonded as the linker to the substrate coated with the single-walled carbon nanotube and the antibody capable of capturing an allergy is bonded to the linker is used. Accordingly, a nanobiosensor based on a single-walled carbon nanotube which is capable of sensing a target allergy-inducing material with high sensitivity in a short time may be fabricated with a simple process.

Further, the present invention provides an allergy detection system comprising the nanobiosensor for detecting allergies as described above; a resistance-sensing unit electrically connected to the nanobiosensor to thus sense a change in the resistance value of the nanobiosensor; and a display unit displaying the change in the resistance value sensed using the resistance-sensing unit.

Further, the present invention provides the nanobiosensor prepared using the above-described method, and also provides an allergy detection system comprising the nanobiosensor, a resistance-sensing unit which is electrically connected to the nanobiosensor and which supplies current to the nanobiosensor to thus sense a change in the resistance value of the nanobiosensor, and a display unit displaying the change in the resistance value sensed using the resistance-sensing unit, thereby detecting allergies using an electrochemical change (see FIG. 2).

The allergy detection system is provided with a nanobiosensor which is capable of sensing a change in resistance value caused when the target allergy-inducing material is bonded to the antibody, thus detecting the target allergy-inducing material with high sensitivity. Accordingly, when the sample is supplied, the target allergy-inducing material comprised in the sample and the antibody are bonded to each other, whereby the change in resistance value in the nanobiosensor is capable of being sensed, the target allergy-inducing material comprised in the sample is capable of being effectively detected by measuring the change in resistance value, and the concentration of the target allergy-inducing material is capable of being easily quantified using a difference in resistance value.

The allergy detection system is provided with a biosensor which is capable of sensing a change in resistance value caused when the target allergy-inducing material is bonded to the antibody, thus detecting the target allergy-inducing material with high sensitivity. Accordingly, when the sample is supplied, the target allergy-inducing material comprised in the sample and the antibody are bonded to each other, whereby the change in resistance value in the nanobiosensor is capable of being sensed, the target allergy-inducing material comprised in the sample is capable of being effectively detected by measuring the change in resistance value, and the concentration of the target allergy-inducing material is capable of being easily quantified using a difference in resistance value.

The allergy detection system is provided with a biosensor designed so as to have two electrodes, comprising a source electrode and a drain electrode, in order to allow continuous resistance-response monitoring for searching for the target allergy-inducing material. Thus, detection is feasible even when the sample including the target allergy-inducing material at a concentration of ng/mL or less is supplied, thereby the allergy-inducing material is capable of being detected with high sensitivity. In addition, since the allergy detection system is capable of being embodied so as to have a compact size, the allergy detection system may be easily carried. Further, various allergy-inducing materials that are present in food may be effectively sensed depending on the type of antibody provided in the nanobiosensor. In particular, since vicilin-based proteins, conglutin-based proteins, or glycinin-based proteins, which are peanut-allergy-inducing proteins, are capable of being effectively detected, the allergy detection system is capable of being effectively used as a peanut-allergy-detection system for detecting a peanut allergy.

Hereinafter, the present invention will be described in more detail with reference to Examples.

The Examples that are presented are for illustrative purposes only in the present invention, and are not intended to limit the scope of the present invention.

### EXAMPLE

(1) Material

An Ara h1 protein, which is a kind of vicilin protein inducing a peanut allergy, and anti-vicilin antibodies or anti-Ara h1 antibodies (anti-Staphylococcus aureus antibodies) were purchased from INDOOR Biotechnologies, Inc., a US company.

Single-walled carbon nanotubes (SWCNT) with a purity of 95% or more were purchased from Chengdu Organic Chemicals company (Chengdu, China). N,N-dimethylformamide (DMF) was purchased from Daejung Inc. (Siheung, South Korea).

1-pyrene-butanoic acid succinimidyl ester was purchased from Life Technologies (NY, USA).

The anti-vicilin antibodies or anti-Ara h1 antibodies (anti-Staphylococcus aureus antibodies) were diluted to a concentration of 0.003 mg/mL with a carbonate-bicarbonate buffer solution before use. Other reagents were of analytical grade.

(2) Antigen-Antibody Reaction with Respect to Enzyme-Linked Immunosorbent Assay (ELISA)

The antigen-antibody reaction depending on the concentration of the Ara h1 protein was performed using the enzyme-linked immunosorbent assay.

In order to analyze the immune specificity, the Ara h1 protein of 20,000 ng/mL was diluted 1,000 times with a PBS buffer solution to use. A color development reaction was performed, followed by detection using a 405 nm microplate reader (Synergy H1 hybrid reader, Seoul, South Korea). The microplate was incubated for 30 minutes in a dark room at room temperature. After 30 minutes, the color development reaction was detected at 405 nm. The color development reaction was detected using a difference in absorbance. The value obtained by subtracting the absorbance before incubation from the absorbance at an elapsed time of 30 minutes was set as the difference in absorbance.

(3) Fabrication of Mask and Nanobiosensor Platform

A mask for forming a gold electrode on a nanobiosensor platform was prepared before fabricating the nanobiosensor platform. A pattern of gold deposition was provided as an electrode on the platform so that a maximum of six measurements is achieved in a single treatment. The distance between the gold deposits was set to about 0.1 cm. Gold was deposited on the surface of a chromium (Cr)-doped silicon substrate using an e-beam evaporator (SRN-110-1505-R2, Sorona Inc., Pyeongtaek, South Korea) under the condition of vacuum pressure of 4.0×10⁻⁶ torr according to the designed mask.

(4) Fabrication of SWCNT-Based Nanobiosensor

For the noncovalent bonding functionalization of multi-type SWCNTs, the biosensor platform assembled with the SWCNT was reacted with a 6 mM PBSE mixed solution (9.8 mg PBSE and 5 mL DMF) as a linker, and washing was performed using a pure dimethyl formaldehyde (DMF) organic solvent and distilled water at room temperature in order to remove PBSE (1-pyrene-butanoic acid succinimidyl ester) remaining on the surface of the biosensor.

An anti-Ara h1 antibody at a concentration of 4 mg/mL was centrifuged at 12,000 rpm for 20 seconds and was diluted 1,000 times by adding a carbonate-bicarbonate buffer solution (pH 9.3) thereto. The anti-Ara h1 antibody was immobilized on the surface of the multi-type SWCNTs by exposing the antibody to the PBSE linker so as to form a covalent bond with the PBSE linker on the multi-type SWCNTs and performing a reaction overnight at 4° C. The nanobiosensor in which the antibody was immobilized was washed with a PBS buffer solution (pH 7.4). The resistance value of the SWCNT nanobiosensor in which the antibody was immobilized was measured using a potentiostat. The resistance values were measured for each step of the fabrication of the SWCNT nanobiosensor.

(5) Detection of Peanut-Allergy-Inducing Proteins Using a SWCNT Nanobiosensor in Which an Antibody is Immobilized

20 μL of a mixed solution containing peanut-allergy-inducing proteins was dropped on a SWCNT nanobiosensor in which an antibody was immobilized (SWCNT+linker+pAb), and was reacted at room temperature for 30 minutes, thus inducing specific bonding between an Ara h1 protein and the antibody. For a control group, 20 μL of a PBS buffer solution was dropped on a SWCNT nanobiosensor in which an antibody was immobilized to thus perform a reaction. Linear sweep voltammetry measurement after the reaction was performed using a potentiostat. In each treatment, the slope of the current/voltage curve ranging from 0 to 0.1 V was measured using linear regression analysis with a resistance value obtained by inversely calculating current/voltage values. A resistance difference (ΔR) was calculated using the following Equation.

ΔR=(R₁−R₀)/R₀ [Equation]

(R₀=initial resistance after a linker is immobilized, and R₁=final resistance after an Ara h1 protein is immobilized).

<Conclusion>

(1) Analysis of Specificity of Antibody

An enzyme-linked immunosorbent assay was performed in order to determine the specific bonding ability of the anti-Ara h1 protein antibody and the Ara h1 protein.

The enzyme-linked immunosorbent assay was performed using an enzyme-free primary antibody and a secondary antibody bonded to an enzyme. When the enzyme is reacted with a substrate, a color development reaction may be induced between the enzyme bonded to the secondary antibody and the substrate to thus generate a fluorescent compound. Overall, the concentration of the specified peanut-allergy-inducing protein was 0 to 1,000 ng/mL.

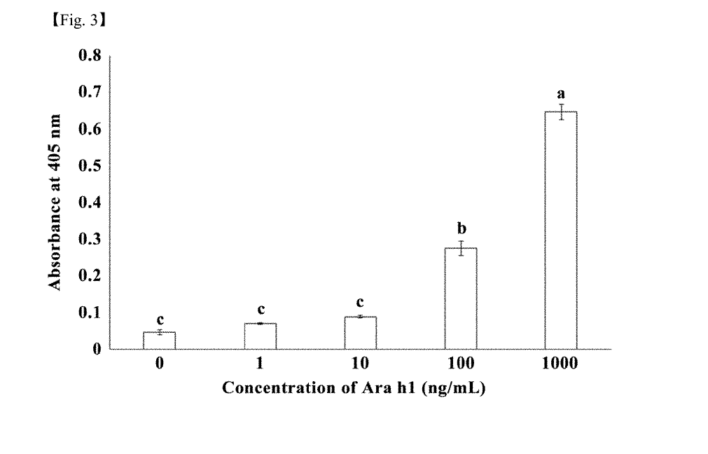

When the peanut-allergy-inducing proteins at different concentrations were introduced into the nanobiosensor as described above, the specificity of the antibody was determined. From FIG. 3, it can be found that the resistance value of the antibody is changed in the case of the sample containing the peanut-allergy-inducing protein at a concentration of ng/mL level. Accordingly, it was confirmed that the antibody is appropriate for the detection of Ara h1, which is the target allergy-inducing protein.

(2) Immobilization of Antibody on SWCNT Nanobiosensor Platform

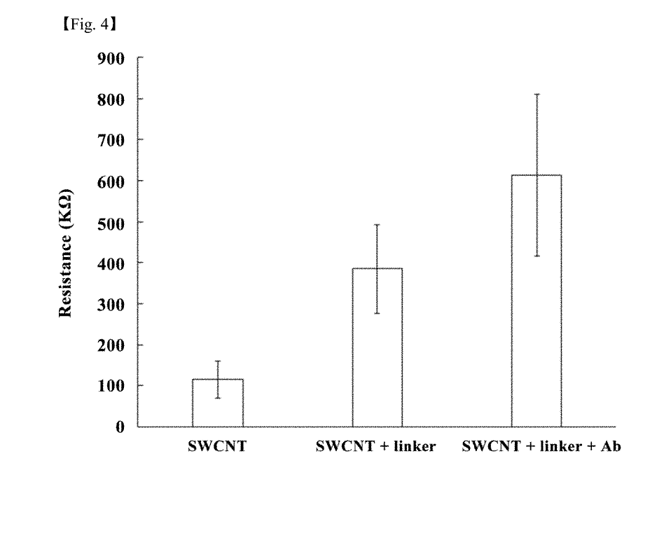

Immobilization of the antibody on the SWCNT nanobiosensor platform requires a linker (PBSE) between the surface of the SWCNT and the antibody. Many biological species may be adsorbed on the surface of the SWCNT through noncovalent bonding due to hydrophobicity, Π-Π stacking, and/or an electrostatic interaction. 1-pyrenobutanoic acid succinimidyl ester may be widely used as the linker of the SWCNT. The hydrophobicity of the pyrenyl group of 1-pyrenobutanoic acid and succinimidyl ester makes it possible to reversibly absorb on the hydrophobic outer wall of the SWCNT through a Π-Π stacking interaction. The bonding of the linker to the surface of the SWCNT does not significantly change the resistance value of the SWCNT nanobiosensor platform (see SWCNT+linker in FIG. 4).

The succinimidyl ester is first reacted with another end of 1-pyrenobutanoic acid, and a secondary amine on the surface of the antibody is reacted for nucleophilic substitution under the solvent condition of DMF. Immobilization of the antibody may greatly increase the resistance value of the nanobiosensor platform (see SWCNT+linker+Ab in FIG. 4). An increase in the resistance value of the nanobiosensor reduces the current and induces the accumulation of negative charges from the antibody. The antibody is divided into two regions consisting of a variable region and a constant region. With respect to this, the variable region of the antibody acts as an antigen bonding site, and the constant region is reacted with target microorganisms or molecules. The immobilization of the antibody on the nanobiosensor platform is achieved by forming a covalent bond resulted from a reaction between the succinimidyl ester, which is a part of the linker, and the amino group of the constant region of the antibody. This interaction may significantly change the resistance value of the SWCNT nanobiosensor platform. When the antibody of the anti-peanut-allergy-inducing protein is immobilized on the SWCNT nanobiosensor platform, the current of a SWCNT-electric-field-effect transistor is reduced by measuring the electric current change of the SWCNT nanobiosensor platform. The device current is reduced because the antibody inhibits electron transfer to the CNT after immobilization of the anti-Ara h1 protein antibody. The electrons are provided to the amide group of the amino acid residue, which changes the threshold potential of the CNT and induces a current reduction.

(3) Measurement of Electrical Resistance Change Value Depending on Capture of Peanut-Allergy-Inducing Protein

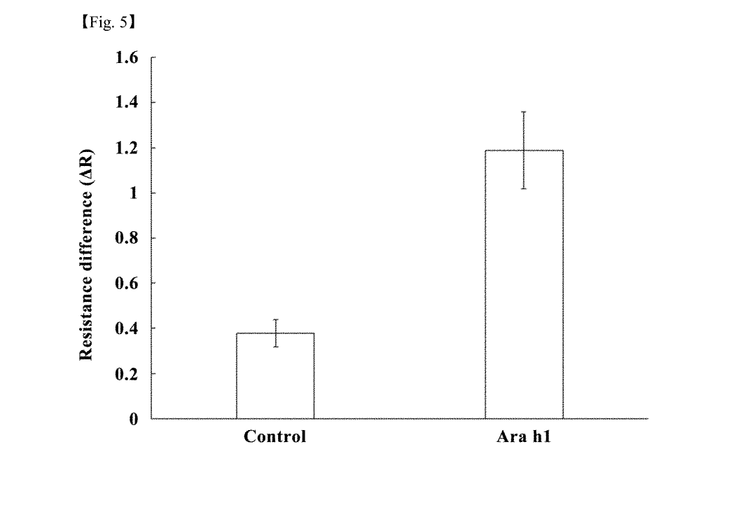

The electrical resistance change of the nanobiosensor platform depending on the presence or absence of the Ara h1 protein was analyzed. As a result, it was found that, when the sample containing the Ara h1 protein was supplied (Ara h1), the difference in the resistance value of the nanobiosensor platform was increased and the resistance value was significantly changed compared to a control group in which a sample containing no Ara h1 protein was supplied (see FIG. 5).

(4) Measurement of Electrical Resistance Change Value for Each Concentration of Peanut-Allergy-Inducing Protein

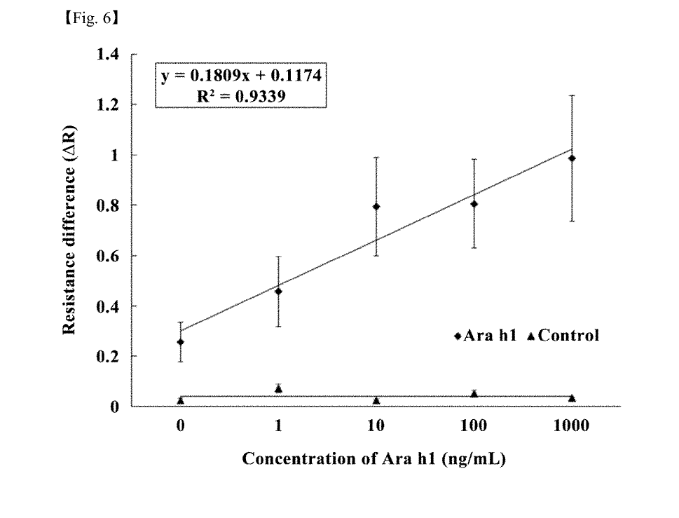

The electrical resistance change value was measured after an Ara h1 protein was supplied to the SWCNT nanobiosensor platform in which the antibody was immobilized while the concentration of the Ara h1 protein was varied. As shown in FIG. 6, the resistance value of the SWCNT nanobiosensor platform was gradually increased as the concentration of the Ara h1 protein was increased, compared to that of a control group. Therefore, it was found that, even when the concentration of the Ara h1 protein is 1,000 ng/mL, the Ara h1 protein is effectively sensed and the resistance value is continuously increased resulting from the bonding of the Ara h1 protein to the nanobiosensor antibody, thereby quantifying the concentration of the Ara h1 protein.

Accordingly, it was found that the nanobiosensor for detecting allergies according to the present invention is capable of being effectively used for the purpose of detection and quantification of peanut-allergy-inducing proteins comprised in food.

1. A nanobiosensor for detecting allergies, comprising:
a substrate;
an electrode layer formed on a portion of an upper side of the substrate;
a single-walled carbon nanotube layer formed on another portion of the upper side of the substrate;
a linker layer formed on the single-walled carbon nanotube layer; and
an antibody which is immobilized and bonded to the linker layer and which is capable of capturing an allergy-inducing material.

2. The nanobiosensor of claim 1, wherein the substrate is a chromium (Cr)-doped silicon substrate.

3. The nanobiosensor of claim 1, wherein the electrode layer comprises gold (Au).

4. The nanobiosensor of claim 1, wherein the linker layer comprises 1-pyrenebutanoic acid succinimidyl ester.

5. The nanobiosensor of claim 1, wherein the antibody captures one or more peanut-allergy-inducing proteins selected from the group consisting of a vicilin-based protein, a conglutin-based protein, and a glycinin-based protein.

6. A method of fabricating a nanobiosensor for detecting allergies, the method comprising:
(a) forming an electrode pattern on a substrate;
(b) forming a single-walled carbon nanotube layer by applying a mixed solution containing a single-walled carbon nanotube on the substrate on which the electrode pattern is formed;
(c) forming a linker layer by applying 1-pyrenebutanoic acid succinimidyl ester on the single-walled carbon nanotube layer; and
(d) bonding an antibody to the linker layer by applying a mixed solution containing the antibody for capturing an allergy-inducing material on the single-walled carbon nanotube layer on which the linker layer is formed.

7. The method of claim 6, wherein, in (a) the forming the electrode pattern, a gold (Au) electrode pattern is formed using an e-beam evaporation method (e-beam evaporator).

8. The method of claim 6, wherein, in (b) the forming the single-walled carbon nanotube layer, the mixed solution contains the single-walled carbon nanotube at a concentration of 0.1 to 10 mg/mL.

9. The method of claim 6, wherein, in (d) the bonding the antibody to the linker layer, the mixed solution containing the antibody, which is capable of capturing one or more peanut-allergy-inducing proteins selected from the group consisting of a vicilin-based protein, a conglutin-based protein, and a glycinin-based protein, is applied to thus form an antibody layer.

10. The method of claim 6, wherein, in (d) the bonding the antibody to the linker layer, the mixed solution containing the antibody at a concentration of 0.001 to 0.01 mg/mL is applied.

11. An allergy detection system comprising:
the nanobiosensor for detecting allergies according to claim 1;
a resistance-sensing unit electrically connected to the nanobiosensor to thus sense a change in a resistance value of the nanobiosensor; and
a display unit displaying the change in resistance value sensed by the resistance-sensing unit.
